(19) United States
(12) Patent Application Publication
(10) Pub. No.: US 2020/0385613 A1
(43) Pub. Date: Dec. 10, 2020

(54) HANDLE COVERS AND HANDLES WITH BIOACTIVE SURFACE COATINGS

(71) Applicants: Dennis Hackemeyer, Forest, VA (US); Mark Sisson, Forest, VA (US)

(72) Inventors: Dennis Hackemeyer, Forest, VA (US); Mark Sisson, Forest, VA (US)

(21) Appl. No.: 16/879,014

(22) Filed: May 20, 2020

Related U.S. Application Data
(60) Provisional application No. 62/850,785, filed on May 21, 2019.
(60) Provisional application No. 63/000,359, filed on Mar. 26, 2020.

(51) Int. Cl. C09J 7/29; C09J 7/40; C09J 11/04; A61L 2/232; A01N 59/00; A01N 59/20; A01N 59/16; B32B 7/06; B32B 7/12; B32B 27/40; B32B 27/18

(52) U.S. Cl. CPC C09J 7/29; A61L 2101/02; C09J 11/04; A61L 2/232; A01N 59/00; A01N 59/20; A01N 59/16; B32B 7/06; B32B 7/12; B32B 27/40; B32B 27/18; C09J 2301/162; C09J 2301/41; C09J 2400/163; C09J 2423/005; C09J 2423/008; C09J 2475/006; C09J 7/405

(57) ABSTRACT

Certain embodiments described herein are directed to handle covers that can reversibly couple to a handle. In some examples, the handle cover comprises a bioactive material that can kill or inactivate bioorganisms. The bioactive material can be a photocatalyst and may also comprise one or more transition metals. Methods of using the handle covers and handles to reduce spread of infections are also described.

### PRIORITY APPLICATIONS

This application is related to and claims priority to and the benefit of U.S. Application No. 62/850,785 filed on May 21, 2019 and U.S. Application No. 63/000,359 filed on Mar. 26, 2020, the entire disclosure of each of which is hereby incorporated herein by reference for all purposes.

### TECHNOLOGICAL FIELD

Certain configurations described herein are directed to handle covers and handles comprising one or more bioactive materials present on a surface and/or embedded in the handle or handle covers.

### BACKGROUND

Many devices include handles which are gripped during use. Different users can transfer germs or other materials to the handle, which can then be transferred to a subsequent user.

### SUMMARY

Certain aspects described herein are related to handle covers that can be coupled to handle of an article. The handle cover can comprise one or more of a surface coating, a coating with an embedded material or both. The exact configuration of the handle cover can depend, at least in part, on the article comprising the handle. Various examples are described to illustrate some of the many different configurations of the handle covers. If desired, a handle itself may comprise a surface coating with a bioactive material such that no handle cover is needed.

In an aspect, a handle cover comprises a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating.

In certain examples, the bioactive material comprises at least one of titanium, silver, copper and zinc. In other examples, the carrier support material comprises a polyurethane. In some embodiments, the bioactive material comprises a photocatalyst comprising titanium, and wherein the bioactive material comprises at least one additional transition metal. In some instances, the handle cover comprises a second bioactive material embedded in the polyurethane of the carrier support material. In some examples, the bioactive material and the second bioactive material comprise different transition metals. In certain embodiments, the first adhesive layer comprises a residue free adhesive. In other examples, the substrate material comprises a circular shape with memory. In some instances, the handle cover is optically transparent. In other examples, the substrate comprises a polyolefin.

In an additional aspect, a handle comprises an adhesive layer coupled to a handle substrate, a carrier support material coupled to the adhesive layer, and a surface coating coupled to the carrier support material, the surface coating comprising a bioactive material to inactivate or kill bioorganisms that contact the surface coating.

In some embodiments, the bioactive material comprises at least one of titanium, silver, copper and zinc. In other examples, the carrier support material comprises a polyurethane. In certain instances, the bioactive material comprises a photocatalyst comprising titanium, and wherein the bioactive material comprises at least one additional transition metal. In other examples, the handle comprises a second bioactive material embedded in the polyurethane of the carrier support material. In some examples, the bioactive material and the second bioactive material comprise different transition metals. In certain embodiments, the adhesive layer comprises a residue free adhesive. In other embodiments, the handle comprises a tear off coupled to the surface coating, wherein the tear off comprises a second adhesive layer coupled to the surface coating, a second carrier support material coupled to the second adhesive layer, and a second surface coating coupled to the second carrier support material, the second surface coating comprising a bioactive material to inactivate or kill bioorganisms that contact the second surface coating. In some embodiments, the adhesive layer, the carrier support material and the surface coating are optically transparent. In other embodiments, the handle substrate comprises a polyolefin.

In another aspect, a handle cover comprises a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover.

In some examples, the bioactive material comprises at least one of titanium, silver, copper and zinc. In certain embodiments, the carrier support material comprises a polyurethane. In other embodiments, the bioactive material comprises a photocatalyst comprising titanium, and wherein the bioactive material comprises at least one additional transition metal. In some examples, the handle cover comprises a second bioactive material embedded in the polyurethane of the carrier support material. In some instances, the bioactive material and the second bioactive material comprise different transition metals. In certain examples, the first adhesive layer comprises a residue free adhesive. In other examples, substrate material comprises a circular shape with memory. In some examples, the handle cover is optically transparent. In other examples, the substrate comprises a polyolefin.

In an additional aspect, a handle comprises an adhesive layer coupled to a handle substrate, and a carrier support material coupled to the adhesive layer, wherein the carrier support material comprising an embedded bioactive material to inactivate or kill bioorganisms that contact the handle.

In another aspect, a shopping cart handle cover comprises a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the shopping cart handle cover is sized and arranged to wrap around at least a portion of a handle of a shopping cart.

In another aspect, a shopping cart handle cover comprises a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the shopping cart handle cover is sized and arranged to wrap around at least a portion of a handle of a shopping cart.

In an additional aspect, a shopping cart comprises a handle and a receptacle coupled to the handle, wherein the handle of the shopping cart comprises an adhesive layer coupled to the handle, a carrier support material coupled to the adhesive layer, and a surface coating coupled to the carrier support material, the surface coating comprising a bioactive material to inactivate or kill bioorganisms that contact the surface coating.

In another aspect, a shopping cart comprises a handle and a receptacle coupled to the handle, wherein the handle of the shopping cart comprises an adhesive layer coupled to the handle, and a carrier support material coupled to the adhesive layer, wherein the carrier support material comprising an embedded bioactive material to inactivate or kill bioorganisms that contact the handle.

In an additional aspect, a vehicle handle cover comprises a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the vehicle handle cover is sized and arranged to wrap around at least some portion of a handle of the vehicle.

In another aspect, a vehicle handle cover comprises a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the vehicle handle cover is sized and arranged to wrap around at least a portion of a handle of the vehicle.

In an additional aspect, a vehicle comprises a handle configured to be grasped by an operator or passenger of the vehicle, the handle comprising an adhesive layer coupled to the handle, a carrier support material coupled to the adhesive layer, and a surface coating coupled to the carrier support material, the surface coating comprising a bioactive material to inactivate or kill bioorganisms that contact the handle.

In another aspect, a vehicle comprises a handle configured to be grasped by an operator of passenger of the vehicle, the handle comprising an adhesive layer coupled to the handle, and a carrier support material coupled to the adhesive layer, wherein the carrier support material comprises an embedded bioactive material to inactivate or kill bioorganisms that contact the handle.

In an additional aspect, a stair rail cover comprises a release liner, a first adhesive layer coupled to the release liner, a substrate coupled to the first adhesive layer, a second adhesive layer coupled to the substrate, a carrier support material coupled to the second adhesive layer; and a surface coating coupled to the carrier support material, the surface coating comprising a bioactive material to inactivate or kill bioorganisms that contact the surface coating, wherein the stair rail handle cover is sized and arranged to wrap around at least some portion of a rail of the stair rail.

In another aspect, a stair rail cover comprises a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the stair rail cover is sized and arranged to wrap around at least a portion of a rail of the stair rail.

In an additional aspect, a stair rail comprises an adhesive layer coupled to the stair rail, a carrier support material coupled to the adhesive layer, and a surface coating coupled to the carrier support material, the surface coating comprising a bioactive material to inactivate or kill bioorganisms that contact the stair rail.

In another aspect, a stair rail comprises an adhesive layer coupled to the stair rail, and a carrier support material coupled to the adhesive layer, wherein the carrier support material comprises an embedded bioactive material to inactivate or kill bioorganisms that contact the stair rail.

In another aspect, a door handle cover comprises a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the door handle cover is sized and arranged to wrap around at least some portion of a handle of a door.

In an additional aspect, a door handle cover comprises a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the vehicle handle cover is sized and arranged to wrap around at least a portion of a handle of a door.

In another aspect, a door handle comprises an adhesive layer coupled to the door handle, a carrier support material coupled to the adhesive layer, and a surface coating coupled to the carrier support material, the surface coating comprising a bioactive material to inactivate or kill bioorganisms that contact the door handle.

In an additional aspect, a door handle comprises an adhesive layer coupled to the door handle, and a carrier support material coupled to the adhesive layer, wherein the carrier support material comprises an embedded bioactive material to inactivate or kill bioorganisms that contact the door handle.

In another aspect, a sports equipment handle cover comprises a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the door handle cover is sized and arranged to wrap around at least some portion of a handle of a sporting equipment article.

In an additional aspect, a sports equipment handle cover comprises a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the vehicle handle cover is sized and arranged to wrap around at least a portion of a handle of a sporting equipment article.

In another aspect, a sports equipment article comprising a handle comprises an adhesive layer coupled to the handle, a carrier support material coupled to the adhesive layer, and a surface coating coupled to the carrier support material, the surface coating comprising a bioactive material to inactivate or kill bioorganisms that contact the handle.

In another aspect, a sports equipment article comprising a handle comprises an adhesive layer coupled to the handle, and a carrier support material coupled to the adhesive layer, wherein the carrier support material comprises an embedded bioactive material to inactivate or kill bioorganisms that contact the handle.

In another aspect, a method comprises placing one or more of the handle covers or handles described herein onto another article or device to facilitate transfer of infectious organisms, infectious virus, infectious viral agents or infectious viral particles from a human to the placed handle cover or handle. In certain embodiments, the placed handle cover or handle comprises a bioactive material to kill or inactivate the transferred infectious organisms, infectious virus, infectious viral agents or infectious viral particles. In other embodiments, the virus that is transferred and inactivated is a coronavirus.

In an additional aspect, a method comprises reducing community spread of an infection by placing one or more of the handle covers or handles described herein onto another article or device to facilitate transfer of infectious organisms, infectious virus, infectious viral agents or infectious viral particles from a user to the placed handle cover or handle. In some examples, the placed handle cover or handle comprises a bioactive material to kill or inactivate the transferred infectious organisms, infectious virus, infectious viral agents or infectious viral particles so successive humans touching the placed handle cover or handle do not become infected by the infectious organisms, infectious virus, infectious viral agents or infectious viral particles. In some examples, the virus that is transferred and inactivated is a coronavirus.

In another aspect, a method of treating a human infected with an infectious organism, an infectious virus, an infectious viral agent or an infectious viral particle comprises administering to the infected human a therapeutic to treat the infection, and reducing spread of the infection from the infected human to third parties by placing one or more of the handle covers or handles described herein onto another article or device facilitate transfer of the infectious organisms, infectious virus, infectious viral agents or infectious viral particles from the infected human to the placed handle cover or handle. In some examples, the placed handle cover or handle comprises a bioactive material to kill or inactivate the transferred infectious organisms, infectious virus, infectious viral agents or infectious viral particles so successive humans touching the placed article do not become infected by the infectious organisms, infectious virus, infectious viral agents or infectious viral particles. In certain examples, the therapeutic is an antimicrobial agent or an antiviral agent or comprises an antimicrobial agent and an antiviral agent.

In an additional aspect, a kit comprises a therapeutic to treat human infected with an infectious organism, an infectious virus, an infectious viral agent or an infectious viral particle, and a handle cover or handle comprising a bioactive material to kill or inactivate any infectious organisms, infectious virus, infectious viral agents or infectious viral particles transferred from the infected human so successive humans touching the handle cover or handle do not become infected by the infectious organisms, infectious virus, infectious viral agents or infectious viral particles. In certain embodiments, the therapeutic of the kit is an antimicrobial agent or an antiviral agent or an antimicrobial agent and an antiviral agent. In some embodiments, the kit comprises written or electronic instructions for using the therapeutic to treat the infection and using the handle cover or handle to prevent or reduce spread of the infection.

Additional aspects, embodiments, examples and configurations are described in more detail below.

### BRIEF DESCRIPTION OF THE SEVERAL VIEWS OF THE DRAWINGS

FIG. 1 is an illustration showing a handle cover coupled to a handle surface of an article, in accordance with some examples;

FIG. 2 is an illustration showing a bioactive material present in a surface coating, in accordance with some examples;

FIG. 3 is an illustration showing a bioactive material embedded in a carrier support material of a surface coating, in accordance with some examples;

FIG. 4 is an illustration showing a bioactive material present in a surface coating and embedded in a carrier support material, in accordance with some examples;

FIGS. 5A, 5B, 5C, 5D and 5E show shapes for different carrier support materials;

FIG. 6 is an illustration showing a handle comprising a bioactive material, in accordance with some examples;

FIG. 7 is an illustration showing a handle cover with a memory that is retained in a curve shape, in accordance with some embodiments;

FIG. 8 is an illustration of a handheld shopping cart or basket, in accordance with certain examples;

FIG. 9 is an illustration of a shopping cart with wheels, in accordance with certain examples;

FIG. 10 is an illustration of a subway car handle, in accordance with certain examples;

FIG. 11 is an illustration of a stair rail handle, in accordance with some embodiments;

FIG. 12 is an illustration of a door handle, in accordance with certain examples;

FIGS. 13A-13D are illustrations showing sporting equipment with handles, in accordance with some embodiments;

FIG. 14 is a table showing reduction of a coronavirus using an article with a bioactive material; and

FIG. 15 is a table showing reduction of E. coli using an article with a bioactive material

### DETAILED DESCRIPTION

Certain articles are described below in connection with handle covers and handles. The handle may be present on many different devices including, but not limited to, gym equipment, doors, car door handles, train handles, subway car handles, adult novelty devices, shopping carts, stair rails and other handles or articles.

As noted herein, the handle cover is typically configured as a sheet that can couple to and/or wrap around a surface of a handle. In some instances, the handle cover can be integral to the handle, whereas in other examples, the handle cover can be coupled to the handle through an adhesive layer present on the handle cover. The handle cover is typically non-electronic and does not comprise any moving parts but does include one or more bioactive materials as noted in more detail below.

In some examples, the handle covers and handles described herein can comprise a bioactive material that can kill or inactivate bioorganisms. The term “bioorganism” is intended to include, but is not limited to, bacteria, fungi and bacterial and fungal spores as well as any viruses or portions thereof, e.g., any membrane components or other components of the bacteria, fungi or virus that may be secreted. Illustrative bioorganisms that are targeted include gram positive and gram negative bacteria, Staphylococcus, Escherichia coli, Propionibacteria, Corynebacteria, dermobacteria, and micrococci, Tinea, Candida, flu virus, adenoviruses and other bacterial, fungi and viruses. The bioactive material may also be effective to inactivate or render non-toxic secreted proteins and materials such as endotoxins or other toxins.

In certain embodiments, the handle covers and/or handles described herein may comprise one or more surface coatings or layers. In some examples, the surface coating may comprise a bioactive material on an outer surface of the surface coating. In other instances, the surface coating may comprise an embedded bioactive material. In additional examples, the surface coating may comprise a bioactive material on an outer surface of the surface coating and may also comprise an embedded bioactive material. For example, as the bioactive material on the outer surface breaks down or is otherwise removed by contact, the embedded bioactive material may still be present to kill or inactivate bioorganisms. In some examples, the surface coating or layer may comprise a carrier material or support that can receive the bioactive material on its surface or can permit embedding of the bioactive material. While not wishing to be bound by any one configuration, the surface coating is generally a non-transfer surface coating such that no or little material is transferred to a user contacting the surface coating with their hands, fingers or other body part. For example, bioorganisms can be transferred from the user to the surface coating where they are inactivated, killed or oxidized by the bioactive material.

In certain embodiments, the bioactive material in the surface coating may be photoactivatable and/or photorechargeable to permit continuous use and reuse of the article. For example, the surface coating can be exposed to infrared, visible, ultraviolet or light of other wavelengths to activate the bioactive material in the surface coating such that the bioactive material can function as a photocatalyst. For example, the bioactive material may comprise photocatalytic titanium dioxide or other photocatalytic transition metal materials. Once activated, the bioactive material can, for example, oxidize groups or constituents on bioorganisms to inactivate and/or kill them. The bioactive material may be photorechargeable by exposing the bioactive material to additional light for an activation period, e.g., 10 second or more, 20 seconds or more, 30 second or more, 1 minute or more, etc. Reactivation recharges the bioactive material for addition use. While the bioactive material may be exposed to light for a suitable period, actual recharging of the material can occur quickly, e.g., within a few microseconds, milliseconds, etc.

In some configurations, the bioactive material may comprise a metal or a material which can release ions, e.g., within the carrier support. For example, the bioactive material can be a transition metal or a transition metal containing material that includes one or more transition metals which can be present in ionic form and/or complexed with one or more ligands. Without wishing to be bound by any one configuration, the transition metal may be present in different forms in the carrier material including free ions and complexed ions. In some examples, the transition metal may be any one or more of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, and mercury, with non-radioactive transition materials being desirable to use and with ionized forms of the transition metals being desirable for use in some instances. In some instances, the bioactive material may comprise two or more different transition metals each of which can independently be present as free ions or complexed with a ligand or other groups. In other instances, the bioactive material may comprise three or more different transition metals each of which can independently be present as free ions or complexed with a ligand or other groups. Where the bioactive material is a photocatalyst, the bioactive material may comprise one or more transition metals. Where the bioactive material is embedded in a surface coating or a carrier support material, the bioactive material desirably can release transition metal ions which can bind to and/or be taken up by the bioorganisms.

In certain embodiments, the transition metal material can be selected to oxidize constituents or groups present on the bioorganisms to kill or inactivate the bioorganisms. For example, the transition metal or transition metal material may function as a photocatalyst and can be activated by exposure of the surface coating to ultraviolet or visible light. Subsequent to initial use, the transition metal or transition metal material can be photo-recharged by exposing the surface coating to additional ultraviolet or visible light. The exact interval where at least 50% of the material remains in an activated form may vary from a few days to a few weeks or even a few months. At any time, a certain amount of the bioactive material may be present in an active state to photocatalyze the received bioorganisms while some portion of the bioactive material may be present in an inactive state. Recharging may be performed, for example, when the amount of bioactive material in the active state drops below a certain percentage, e.g., 50%, 40%, 30%, 20% or even 10%.

In certain configurations, the bioactive material can also be present on top of the carrier support material as a separate surface coating. For example, the support material may comprise embedded bioactive material and additional bioactive material may be present as a separate surface layer or surface coating on top of the carrier support material comprising the embedded bioactive material. In some cases, bioactive material can migrate from the carrier support material into the outer surface coating or layer to replenish the bioactive material as it is consumed or leaches off. In other instances, the embedded bioactive material does not migrate but can remain active within the carrier support material to kill and/or inactivate bioorganisms. Where a surface coating of bioactive material is deposited on top of a layer comprising the embedded bioactive material in the carrier support material, the surface coating may comprise a transition metal or a transition metal containing material that includes one or more transition metals which can be present in ionic form and/or complexed with one or more ligands. The transition metal bioactive material deposited on top of the carrier support material may be present in different forms in the carrier material including free ions and complexed ions. In some examples, the transition metal present on top of a carrier support material may comprise one or more of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, and mercury, with non-radioactive transition material being desirable to use and with ionized forms of the transition metals being desirable for use in some instances. In some instances, the transition metal bioactive material deposited on top of the carrier support material may comprise two or more different transition metals each of which can independently be present as free ions or complexed with a ligand or other groups. In other instances, the transition metal material deposited on top of the carrier support material may comprise three or more different transition metals each of which can independently be present as free ions or complexed with a ligand or other groups.

In certain examples, the carrier support material of the articles described herein typically is selected to be able to withstand physical contact of the handle covers to surfaces. For example, the carrier material may be a polymeric material that can be disposed on a substrate in a desired shape and using suitable methods, e.g., printing, spraying, coating, dip coating, rolling or using other methods. As noted herein, a bioactive material of the surface coating can be present on or in the carrier material (or both) and used to inactivate or kill bioorganisms. In some embodiments, the carrier support material can be selected such that it retain the bioorganisms within the surface coating, e.g., prevents transfer of the bioorganisms back to a second user contacting the articles. In some embodiments, the carrier material may comprise one or more thermoplastics or one or more thermosetting materials. For example, polyurethanes, polyacrylates, and copolymers comprising polyurethanes, polyacrylates or other polymeric materials that are optically transparent when placed on a substrate can be used. In other instances, the carrier support material may be a polyester, an epoxy resin, a polyimide, a silicone resin, a vinyl ester resin, a polycarbonate, a polyetherimide, a polypropylene, a polyphenylene oxide, a polyphenylene sulfide or other resins or materials that are desirably optically transparent, though the carrier support materials may be opaque or partially opaque if desired.

In certain embodiments, the surface coating may be hard, soft, non-compressible or compressible depending on the end use and configuration of the final article. In some examples, the carrier support material may be elastic and optionally comprise one or more elastomeric materials. For example, upon depression of the article by a user, the article may spring back to an initial position after removal of the depressing force. In some instances, rubber, natural rubber, synthetic rubber or other rubber based materials may be present in the carrier support material.

In other instances, the bioactive material may form clusters on top of the carrier support material with open space present between the clusters. In such instances, it may be desirable to include embedded bioactive material in the carrier support material as well in case bioorganisms do not contact any of the surface clusters of the bioactive material.

In certain embodiments, the articles described herein are typically placed on top of another article or device and can be designed to permit viewing of the underlying article or device. For example, the entire article may be produced using materials which are generally transparent, e.g., over visible wavelength ranges, such that viewing of the underlying article or device is permitted. The article need not transmit 100% of the light but is generally transparent enough so underlying text or other features of the device or article is viewable using the naked eye. Even though the articles can be optically transparent, they may be colored if desired. Alternatively, the articles may be colorless.

In certain embodiments, the layers of the articles may comprise fibers, elastomers or other materials to alter the overall properties of articles. For example, elastomeric fibers may be present to permit depression or compression of the articles during use. Further, additional materials may be present to provide touch indicia such as Braille, raised letters or numbers or other features.

In certain configurations, the articles described herein generally comprise a substrate upon which the surface coating (or other layers) is placed. In certain embodiments, suitable substrates that can be used with the surface coating described herein may be optically transparent, printed or may be opaque if desired. In certain examples, the exact material used in the substrate can vary depending on the intended use environment of the article. In some examples, the substrate may comprise a paper, a fabric, a metal, a non-metal, a plastic, a ceramic, a glass, a fiberglass, a stone, a wood, a rubber, a foam, a textile, cardboard, a vinyl material, concrete, asphalt, leather, suede, a polymeric material or other materials. In embodiments where papers are used, the paper may be acid-free or may be designed to be present in its use environment for a desired period without substantial degradation. In examples where a fabric is used, the fabric may be a woven fabric, a non-woven fabric, a polyester fabric such as, for example, a draw textured yarn (DTY) polyester fabric, a polyester-copolymer fabric and other fabrics commonly used to receive inks and colorants using printing techniques, lithographic techniques or other techniques. For example, polyester DTYs are effective to absorb and retain inks and other colorants. In addition, polyester DTYs can permit even distribution of the inks or colorants to provide desirable indicia. Illustrative DTY' s can be found, for example in U.S. Patent Publication No. 20110008563 filed on Jul. 9, 2009, the entire disclosure of which is incorporated herein by reference. Where the substrate is a ceramic, the ceramic may be, for example, aluminum oxide, yttrium oxide, cerium oxide, beryllia, zirconia, a carbide, a boride, a nitride, a silicide or other ceramic materials. Where the substrate is a glass, the glass may be colored, non-colored, opaque, transparent or may include variable areas having different properties. If desired, the glass may include reinforcing fibers or other materials to strengthen the physical properties of the glass. Where the substrate is a stone, wood, rubber, foam or other material, the material may be porous such that physical indicia can be imparted to the material. If the material is highly porous, then it may be desirable to reduce the porosity of the material by first disposing an agent on the material that can occupy some of the pores of the material. In some embodiments, the substrate may be a plastic material such as, for example, a thermoplastic material or a thermosetting material.

In some examples, the substrate may comprise a polyolefin material that is optically transparent. For example, the substrate material may comprise a polyethylene, a polyethylene copolymer, a polyvinyl chloride, a polyvinyl chloride copolymer, a polypropylene or other polyolefins that are optically transparent at least to some degree. In some embodiments, the polyolefin may be non-porous and be configured to retain the surface coating and optionally other materials on a surface of the substrate. For example, while the surface coating may include some porosity to permit bioorganisms to penetrate into the surface coating, the substrate generally is non-porous or fully consolidated such that surface coating materials do not penetrate into the substrate. Illustrative substrates are commercially available from many different sources including, but not limited to, those from the PhotoTex® Group Inc. (Boardman, Ohio), Fusion Digital (Washington, Utah), Yupo (Chesapeake, Va.), Granwell (West Caldwell, N.J.), Superior Fabrics (Pompano Beach, Fla.), Worthen Industries (Nashua, N.H.) and other commercial sources. In some instances, the substrate can be flexible, rigid, semi-rigid, compressible or may have other physical properties as desired.

In some examples, the overall shape and thickness of the various layers may vary as desired and depending on the intended use of the article. In some examples, the carrier support material layer may comprise a thickness, for example, of about 0.5 mm to about 5 mm. Where a surface coating of bioactive material is present on top of the carrier support material layer, the surface coating thickness may be, for example, about 0.1 mm to about 1 mm. The overall thickness of the substrate can vary from about 0.1 mm to about 5 mm. The width and length of the articles depend on the end use of the articles and illustrative values are discussed below. The thickness of the bioactive material can be as little as 1-2 crystals, e.g., 7-10 nm or less.

In certain embodiments, one or more protective layers, materials or coatings may be present on the articles described herein. The protective layer, material or coating may be present between two or more other components of the articles as desired or within any one or more layers. In some examples, the protective layer, material or coating may be present on top of the active surface layer or coating. For example, in applications where the articles are used outside, a UV protective material, color fast material or other materials may be present on top of the active surface coating or mixed with it to protect it. The protective coating, material or layer can be porous to permit bioorganisms to be transferred from a user's hand or other body part to the underlying active surface coating or layer for inactivation, oxidation and/or killing. In other examples, the protective layer or material may be present on top of the substrate and used to render the substrate color fast or protect any ink or other colorant on the substrate from photobleaching, UV degradation or degradation due to other means. Suitable materials for use as a protective layer or coating include, but are not limited to, acrylates such as, for example, trimethylpropaneacrylate, epoxyacrylate, urethaneacrylate and other acrylates. Other polymeric materials including polyolefins, nanoparticles and the like may also be present as protective coatings.

In certain embodiments, the articles described herein typically comprise an adhesive layer between the substrate and a release liner. The adhesive layer can be designed to adhere the article to an underlying device or another article. In some examples, the adhesive can be a residue free adhesive such that removal of the article from the underlying device or article does not leave behind any adhesive on the surface of the underlying device or article. Illustrative adhesives include but are not limited to thermoplastic adhesives and thermosetting adhesives. For example, the adhesive may comprise one or more of adhesives or residue-free adhesives that are commercially available from 3M, Henkel, Shell Chemical Company, Kuraray Company and other commercial suppliers of adhesives. In some examples, the adhesive may comprise rubber or other elastomer and be a residue-free adhesive. In other examples, the adhesive can be a silicon based adhesive such as, for example, an organopolysiloxane adhesive. In some examples, the adhesive can include one or more cross-linkable groups such as, for example, an isocyanate group, an unsaturated hydrocarbon group, a sulfo group, a sulfhydryl group, an alkoxy group, a hydroxyl group, and other groups that can be cross-linked. In some embodiments, the adhesive can be used in combination with a crosslinking agent to facilitate cross-linking and/or curing of the adhesive. In some embodiments, the adhesive can include one or more materials, polymers or copolymers including, but not limited to, styrene block polymers such as, for example, a styrene and styrene/diene copolymer (SBS, SIS, SBR), a styrene/ethylene/butylene copolymer (SEBS) or a styrene/ethylene/propylene/styrene copolymers (SEPS), acrylate copolymers, a polyester urethane copolymer, an ethylene acrylate copolymer, a butyl rubber copolymer; a natural rubber copolymer; an ethylene/propylene copolymer; an ethylene/vinyl acetate copolymers, EPDM/PP, NR/PP, EVA/PVDC and NBR/PP, polyurethanes, polyether esters and polyether amides based copolymers or materials. Additional materials and groups can also be used to prepare the adhesive including, but not limited to, homo- or copolymers of 1,3-butadiene, 2-methyl-1,3-butadiene (isoprene), 1,3-pentadiene, 2-ethyl-1,3-butadiene, 2-propyl-1,3-butadiene, 2-isopropyl-1,3-butadiene, 2-hexyl-1,3-butadiene, 2,3-dimethyl-1,3-butadiene, 2,3-diethyl-1,3-butadiene, 2-methyl-1,3-pentadiene, 2-methyl-1,3-hexadiene, 2-methyl-1,3-octadiene, 2-methyl-1,3-decadiene, 2,3-dimethyl-1,3-pentadiene, 2,3-dimethyl-1,3-hexadiene, 2,3-dimethyl-1,3-octadiene and 2,3-dimethyl-1,3-decadiene, 2-methyl-1,3-cyclopentadiene, 2-methyl-1,3-cyclohectadiene, 2,3-dimethyl-1,3-cyclopentadiene, 2,3-dimethyl-1,3-cyclohexadiene, 2-chloro-1,3-butadiene, 2,3-dichloro-1,3-butadiene, 1-fluoro-1,3-butadiene, 2-chloro-1,3-pentadiene, 2-chloro-1,3-cyclopentadiene and 2-chloro-1,3-cyclohexadiene. In some embodiments, isoprene, polyisoprene or isoprene derivatives or polyisoprene derivatives may also be used in the adhesive. If desired, the adhesive may be a pressure sensitive adhesive. In certain examples, the adhesive can be crosslinkable to the substrate using light, heat, a catalyst, an activator or other suitable materials and/or processes.

In certain configurations, an adhesive layer can be present between any two or more component or layers of the articles. For example, an adhesive layer can be present between the carrier support material and the underlying substrate, between the carrier support material and any bioactive surface coating or between other layers that may be present in the articles.

In some examples, a release liner may be present on a surface of the article that is to be coupled to an underlying article or device. The release liner is typically an inert material, e.g., a paper, plastic, rubber, etc. that is used to cover the adhesive layer prior to use of the article. The release liner may comprise, for example, Kraft paper, clay coated paper, machine glazed paper, a polyethyleneterephthalate film, a polyethylene film, a polypropylene film, and other films produced using polyolefin materials. In use of the article, the release liner is typically peeled away from the article to expose the adhesive layer, and the article is pressed onto a desired surface and retained on the surface through the adhesive layer. The article can then be removed at a later time by mechanical force, using heat or a solvent or through other means.

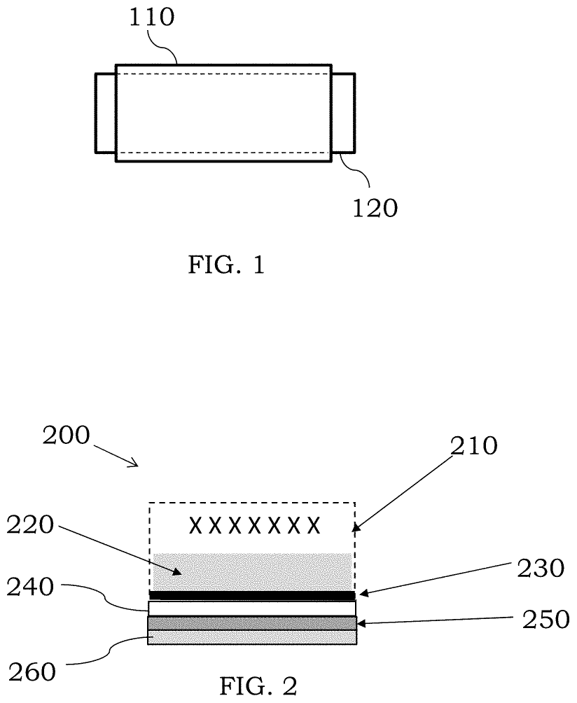

In certain configurations, an illustration showing a handle cover 110 coupled to a handle 120 is shown in FIG. 1. While not shown, the handle 120 is typically coupled to another device or the handle comprises another end or structure. The handle cover 110 can be attached to a surface of the handle 120 through an adhesive layer on the handle cover 110. If desired, a second handle cover (not shown) can also be attached to the handle 120. For example, three, four, five, six or more handle covers can be present.

Referring to FIG. 2, one illustration of certain components and materials that are present in a handle cover is shown. The handle cover 200 comprises a surface coating 210, a carrier support material 220, an adhesive layer 230, a substrate 240, another adhesive layer 250 and a release liner 260. In this illustration, the bioactive material is present in the surface coating 210, which can be sprayed, dip coated, curtain coated, roller coated, printed, brushed or otherwise deposited on the carrier support material 220. If desired, two or more layers of the surface coating 210 can be sprayed onto the support material 220 as individual layers. The carrier support material 220 can be any of those materials mentioned above or other suitable materials. The adhesive layer 230 acts to retain the carrier support material 220 to the underlying substrate 240. The adhesive layer 250 retains the article 200 to a surface of the handle when in use. The adhesive layers 230, 250 can comprise the same or different materials as desired and may be any of the illustrative adhesive materials mentioned herein or other suitable adhesive materials. The substrate 240 is typically optically transparent and may be any of those illustrative materials discussed herein. While certain layers are shown in FIG. 2 as comprising the same thickness, this arrangement is not required or even typical. The adhesive layers 230, 250 tend to be much thinner than the substrate 240 or the carrier support material 220. The bioactive material in the surface coating 210 typically comprises one or more transition metals or transition metal materials including, but not limited to, those comprising titanium, zinc, copper, silver or other transition metals mentioned herein. The bioactive material in the surface coating 210 may be present in ionic form, chelated or bound to other groups or both. In some instances, the bioactive material of the surface coating 210 can be photo-activated by exposure to ultraviolet light or visible light (or both) and can be photo-recharged upon re-exposure to ultraviolet light or visible light (or both). In other examples, the bioactive material in the surface coating 210 can function as a photocatalyst to kill or inactivate bioorganisms that contact the surface coating 210.

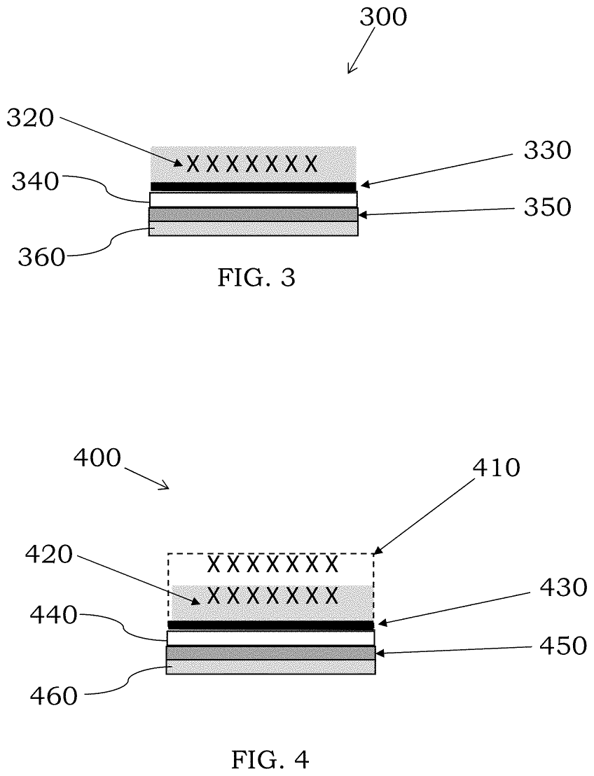

In certain configurations, other material arrangements for the handle covers are also possible. Referring to FIG. 3, another illustration of certain components and materials that are present in a handle cover is shown. The handle cover 300 comprises a surface coating 320 comprising a carrier support material and a bioactive material, an adhesive layer 330, a substrate 340, another adhesive layer 350 and a release liner 360. In this illustration, the bioactive material is embedded within the carrier support material that is present in a surface coating 320, which can be sprayed, dip coated, curtain coated, roller coated, printed, brushed or otherwise deposited on the underlying adhesive layer 330. The carrier support material in the coating 320 can be any of those materials mentioned above or other suitable materials. The adhesive layer 330 acts to retain the carrier support material 320 to the underlying substrate 340. The adhesive layer 350 retains the article 300 to a surface of the handle when in use. The adhesive layers 330, 350 can comprise the same or different materials as desired and may be any of the illustrative adhesive materials mentioned herein or other suitable adhesive materials. The substrate 340 is typically optically transparent and may be any of those illustrative materials discussed herein. While certain layers are shown in FIG. 3 as comprising the same thickness, this arrangement is not required or even typical. The adhesive layers 330, 350 tend to be much thinner than the substrate 340 or the surface coating 320. The bioactive material embedded in the carrier support material present in the surface coating 320 typically comprises one or more transition metals or transition metal materials including, but not limited to, those comprising titanium, zinc, copper, silver or other transition metals mentioned herein. The bioactive material in the surface coating 320 may be present in ionic form, chelated or bound to other groups or both. In some instances, the bioactive material of the surface coating 320 can be photo-activated by exposure to ultraviolet light or visible light (or both) and can be photo-recharged upon re-exposure to ultraviolet light or visible light (or both). In some examples, the bioactive material in the surface coating 320 can function as a photocatalyst to kill or inactivate bioorganisms that contact the surface coating 320.

Referring to FIG. 4, an illustration is shown where a bioactive material is present in both a surface coating and is embedded in a carrier support material that underlies the surface coating. The handle cover 400 comprises a surface coating 410 with a bioactive material, a carrier support material 420 (with a bioactive material), an adhesive layer 430, a substrate 440, another adhesive layer 450 and a release liner 460. In this illustration, the bioactive material is present in the surface coating 410, which can be sprayed, dip coated, curtain coated, roller coated, printed, brushed or otherwise deposited on the carrier support material 420. In addition, the carrier support material 420 also comprises an embedded bioactive material which may be the same or may be different than the bioactive material of the surface coating 410. The carrier support material 420 can be any of those materials mentioned above or other suitable materials. The adhesive layer 430 acts to retain the carrier support material 420 to the underlying substrate 440. The adhesive layer 450 retains the article 400 to the handle when in use. The adhesive layers 430, 450 can comprise the same or different materials as desired and may be any of the illustrative adhesive materials mentioned herein or other suitable adhesive materials. The substrate 440 is typically optically transparent and may be any of those illustrative materials discussed herein. While certain layers are shown in FIG. 4 as comprising the same thickness, this arrangement is not required or even typical. The adhesive layers 430, 450 tend to be much thinner than the substrate 440 or the carrier support material 420. The bioactive material in the surface coating 410 and in the carrier support material 420 typically comprises one or more transition metals or transition metal materials including, but not limited to, those comprising titanium, zinc, copper, silver or other transition metals mentioned herein. The bioactive material in the surface coating 410 and in the carrier support material 420 may be present in ionic form, chelated or bound to other groups or both. In some instances, the bioactive material of the surface coating 410 and the carrier support material 420 can be photo-activated by exposure to ultraviolet light or visible light (or both) and can be photo-recharged upon re-exposure to ultraviolet light or visible light (or both). In some instances, the bioactive material in the surface coating 410 and in the carrier support material 420 can independently function as a photocatalyst to kill or inactivate bioorganisms that contact the handle cover 400.

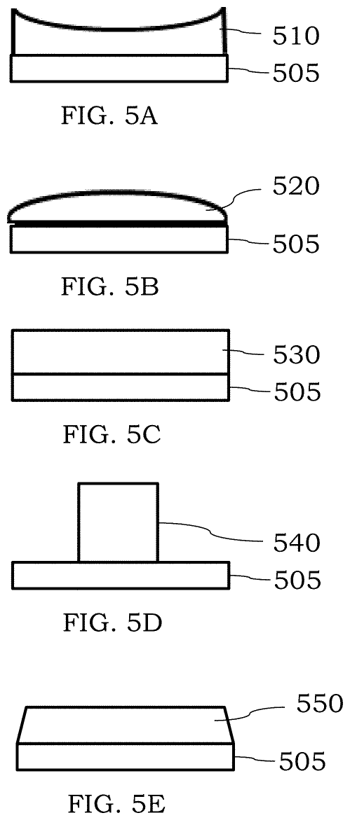

While the carrier support material is shown in FIG. 2-4 as being generally planar or flat, this shape is not required. For example, a concave shape 510 (FIG. 5A), an half-ellipse shape 520 (FIG. 5B), a rectangular shape 530 (FIG. 5C), a square shape 540 (FIG. 5D), a trapezoidal shape 550 (FIG. 5E) or other geometric shapes for the carrier support layer present on a substrate 505 could instead be present and used to produce the handle covers and handles described herein.

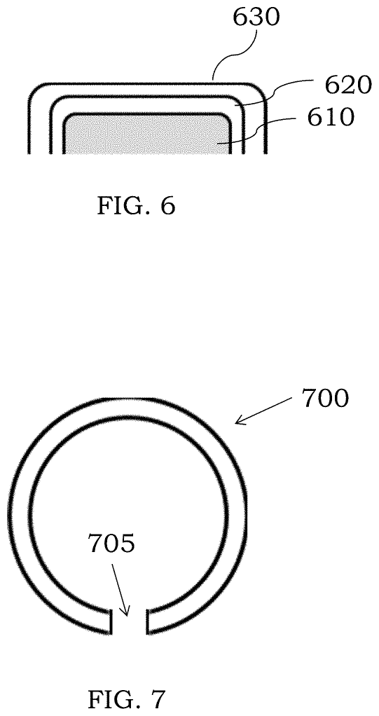

In some examples, the materials described herein can instead be present as a coating on a handle. For example, the substrate layers described in connection with FIGS. 2-4 could be a handle itself, and the various layers may be deposited directly on the handle so no separate handle cover is present. If desired, the handle may comprise a bioactive material and a handle cover can also be used with the handle. Referring to FIG. 6, a cross-section of a handle is shown where the handle comprises an underlying handle substrate 610, an adhesive layer 620 and a carrier support material 630. A surface coating can be present on the carrier support material 630 if desired. The carrier support material 630, the surface coating or both may comprise a bioactive material as described herein. If desired, the adhesive layer 620 can be omitted and the carrier support material 630 can be applied directly to the handle substrate 610.

In certain examples, the handle cover substrate may have a “memory” which acts to retain the handle cover in a desired shape or arrangement. One illustration is shown in FIG. 7, where the handle cover 700 comprises a generally circular arrangement with an opening 705 to permit placement of the cover 700 around a handle. One or more of the layers of the cover 700 may have the memory to retain the cover in the shape even where the handle cover 700 is not coupled to a handle. For example, the substrate of the handle cover may be formed into a desired shape, e.g., using molding, thermoforming or other means, to provide a memory to the cover 700. Alternatively, the cover 700 can be present as a planar sheet which can be wrapped around at least some portion of a handle.

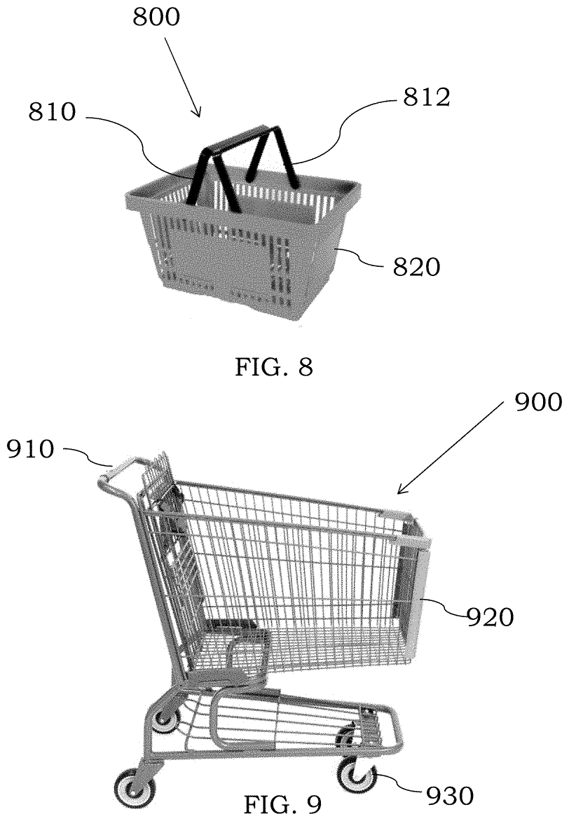

In certain embodiments, the handle cover can be used on a handheld shopping cart as shown in FIG. 8 where the shopping cart 800 includes handles 810, 812 and a receptacle or basket 820. For example, a handle cover can be placed on one or both of handles 810, 812. The handle cover may comprise a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the shopping cart handle cover is sized and arranged to wrap around at least a portion of one of the handles 810, 812 of the shopping cart 800. In other instances, the handle cover used with the shopping cart 800 may comprise a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the shopping cart handle cover is sized and arranged to wrap around at least a portion of one of the handles 810, 812 of the shopping cart 800. Alternatively, one or both of the handles 810, 812 may comprise a bioactive material as described herein so no handle cover is present.

In certain embodiments, the handle cover can be used on a shopping cart with wheels as shown in FIG. 9 where the shopping cart 900 includes a handle 910, a receptacle or basket 920 and wheels including wheel 930. For example, a handle cover can be placed on the handle 910. The handle cover may comprise a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the shopping cart handle cover is sized and arranged to wrap around at least a portion of the handle 910 of the shopping cart 900. In other instances, the handle cover used with the shopping cart 900 may comprise a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the shopping cart handle cover is sized and arranged to wrap around at least a portion of the handle 910 of the shopping cart 900. Alternatively, the handle 910 may comprise a bioactive material as described herein so no handle cover is present.

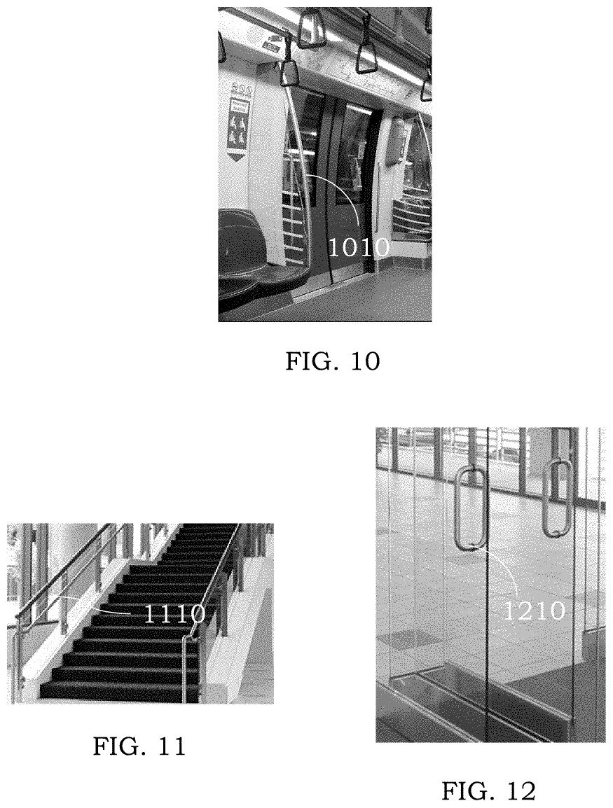

In certain embodiments, the handle covers can be used with handles present in vehicles such as, for example, subway car handles as shown in FIG. 10. For example, a handle cover placed on the handle 1010 may comprise a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the subway car handle cover is sized and arranged to wrap around at least a portion of the handle 1010. In other instances, the handle cover used with the subway car handle 1010 may comprise a release liner, a first adhesive layer coupled to the release liner, a substrate coupled to the first adhesive layer, a second adhesive layer coupled to the substrate, and a carrier support material coupled to the second adhesive layer, and the carrier support material comprising embedded bioactive material to inactivate or kill bioorganisms that contact the handle cover, wherein the subway car handle cover is sized and arranged to wrap around at least a portion of the handle 1010. Alternatively, the handle 1010 may comprise a bioactive material as described herein so no handle cover is present. If desired, a handle cover could instead be placed on one or more of the hanging subway car handles or could also be placed on one or more of the hanging subway car handles. In addition, the handle covers could instead be used on a passenger train handles, airplane handles, bus handles or other automotive or transport vehicles including those powered by gasoline, electricity, diesel fuel, fuel cells, batteries or other power sources.

In certain embodiments, the handle covers described herein can be placed on stair rail 1110 as shown in FIG. 11. In some examples, the stair rail cover comprises a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the stair rail handle cover is sized and arranged to wrap around at least some portion of a rail of the stair rail. In other instances, the stair rail cover comprises a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the stair rail cover is sized and arranged to wrap around at least a portion of a rail of the stair rail. If desired, a plurality of rail covers can be placed along the stair rail to cover substantially all of the stair rail with a cover. Alternatively, the stair rail 1110 may comprise a bioactive material as described herein so no rail cover is present.

In other embodiments, the handle covers described herein can be used with door handle. For example, a door handle cover can be placed on a door handle 1210 as shown in FIG. 12. In some examples, a door handle cover comprises a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the door handle cover is sized and arranged to wrap around at least some portion of a handle of a door. In other examples, a door handle cover comprises a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the vehicle handle cover is sized and arranged to wrap around at least a portion of a handle of a door. Alternatively, the door handle 1210 may comprise a bioactive material as described herein so no door handle cover is present.

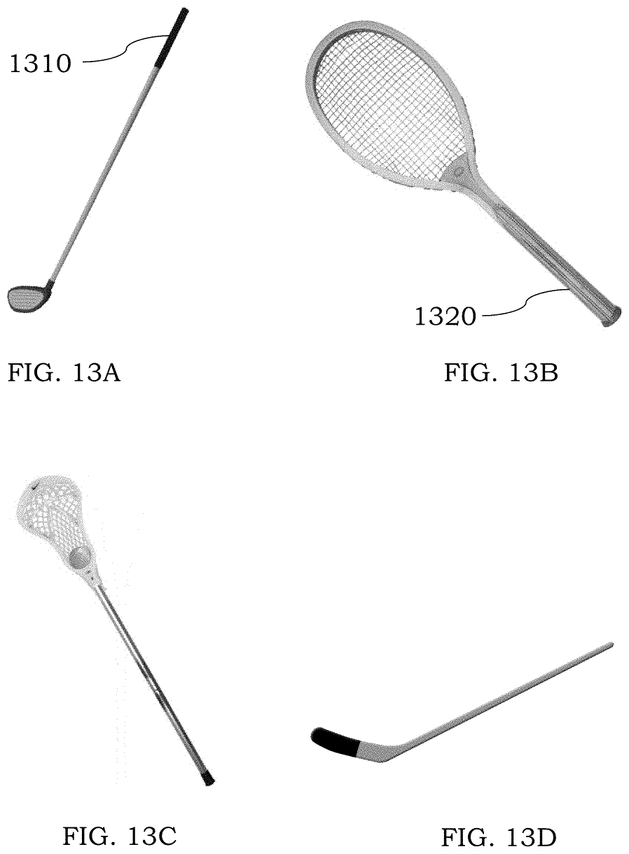

In some examples, the handle covers described herein can be used on handles of sporting equipment including, but not limited to, a gold club handle 1310 (FIG. 13A), a tennis racket handle 1320 (FIG. 13B), a lacrosse stick handle 1330 (FIG. 13C) or a hockey stick handle 1340 (FIG. 13D). The cover used with a sporting equipment article may comprise, for example, a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating, wherein the door handle cover is sized and arranged to wrap around at least some portion of a handle of a sporting equipment article. In other instances, the cover used with the sporting equipment article may comprise a release liner, a first adhesive layer coupled to the release liner, a substrate coupled to the first adhesive layer, a second adhesive layer coupled to the substrate, and a carrier support material coupled to the second adhesive layer, the carrier support material comprising embedded bioactive material to inactivate or kill bioorganisms that contact the handle cover, wherein the vehicle handle cover is sized and arranged to wrap around at least a portion of a handle of the sporting equipment article. Alternatively, the handle itself may comprise a bioactive material so that the handle cover can be omitted.

The handle covers described herein can also be used in other devices or articles which comprise a handle including adult novelty devices, e.g., sex toys, writing utensils, silverware, glasses, cups, mugs and other items including a handle or surface that is gripped by a human hand during use.

The articles and various layers described herein can also be used with additional materials including primers, e.g., titanium dioxide primer layers, colorants, inks, luminescent coatings, surfactants and other materials as desired. Crosslinkers such as amides or other materials can also be used to facilitate rapid curing of the layers or the layers can be cured without the use of any crosslinkers. In some embodiments, one or more of a halogenated phenol, a phenoxy phenol, a hydroxyphenyl ether, a halogenated phenoxy, e.g., fluorinated, chlorinated or brominated phenoxy compounds, polyhexamethylene biguanide (PHMD), PHMD chloride, PHMD fluoride, PHMD bromide, PHMD hydrochloride, Microban® materials, halogenated phenols such as, for example, 5-chloro-2-(2,4-dichlorophenoxy) phenol, chloro-2-(2,4-dichloro)phenol, and chloro-2-(2,4-dichlorophenoxy)phenol, Triclosan, Irgansan DP300, CH3635, Ster-zac, Lexol 300, trichloro-2-hydroxydiphenyl ether, plant oils such as, for example, tea tree oil, mint oil, leleshwa oil, sandalwood oil, clove oil, lavender oil, nigella sativa (Black cumin) oil, onion, garlic and combinations thereof can also be present in the surface coating or carrier support material or both. In some instances, the surface coating, carrier support material or both may include one or more materials commercially available from Environ (Rochester Hills, Mich.), Microban (Huntersville, N.C.), or Oxititan (Pompano Beach, Fla.) or other producers of antimicrobial ingredients.

Certain embodiments described herein can be used in methods and device to reduce infections and community spread of infections. The methods can desirably use one or more of the handle covers or handles described herein.

In some embodiments, a method of reducing infections comprises placing one or more of the handle covers or handles described herein onto another article or device to facilitate transfer of infectious organisms, infectious virus, infectious viral agents or infectious viral particles from a user to the handle covers or handles or surface. A bioactive material on the placed handle cover or handle can inactivate and/or kill the infectious virus, infectious viral agents or infectious viral particles to prevent infection of a subsequent user who contacts the handle cover or handle or surface.

In another embodiment, a method of reducing community spread of an infection comprises placing one or more of the handle covers or handles described herein onto another article or device facilitate transfer of infectious organisms, infectious virus, infectious viral agents or infectious viral particles from a user to the handle cover or handle or surface. A bioactive material on the placed handle cover or handle can inactivate and/or kill the infectious virus, infectious viral agents or infectious viral particles to prevent community spread of the infectious virus, infectious viral agents or infectious viral particles.

In another example, a method of treating a person infected with an infection while reducing spread of the infection from the infected person comprises placing a handle covers or handles comprising one or more bioactive materials on a corresponding receptive article and administering to the infected person in need of treatment one or more antiviral drugs, antimicrobial drugs or anti-parasitic drugs or combinations thereof. The drug administration can treat the infected person while the placed handle cover or handle can reduce spread of the infection from the human being treated to third parties. For example, the method can reduce spread by killing or inactivating of infectious organisms, infectious virus, infectious viral agents or infectious viral particles that have been transferred to the article using one or more bioactive materials on the handle cover or handle.

In certain examples, the methods and handle covers or handles described herein can be used to prevent or reduce the spread of a virus including double-stranded DNA viruses, a single-stranded DNA virus, a double-stranded RNA virus, a single stranded RNA virus, a single-stranded RNA retrovirus, a double- stranded DNA retrovirus and other viruses including either double-stranded DNA or RNA or single stranded DNA or RNA or hybrid DNA-RNA nucleic acid. Specific types of viruses include, but are not limited to, a picornavirus, a coronavirus, a rhinovirus, an adenovirus, an enterovirus, an influenza virus, a human parainfluenza virus, a human respiratory syncytial virus, a metapneumovirus, a retrovirus, a norovirus, a rotavirus, a herpes virus, a poxvirus, a reovirus, an orthomyxovirus, a rhabdovirus, a parvovirus and other viruses that can infect mammals such as humans or other animals. As noted in more detail below, the devices are particularly effective at reducing active levels of coronaviruses such as, for example, coronavirus 229E, coronavirus NL63, coronavirus 0C43, coronavirus HKU1, MERS-CoV, SARS-CoV and SARS-CoV-2 (COVID-19). In some instances, at least 95% of the coronavirus transferred to the surface can be killed or inactivated by the bioactive material within 30 minutes after transfer to the surface. In other instances, at least 95% of the coronavirus transferred to the surface can be killed or inactivated by the bioactive material within 60 minutes after transfer to the surface. In some embodiments, at least 95% of the coronavirus transferred to the surface can be killed or inactivated by the bioactive material within 120 minutes after transfer to the surface.

In some instances, at least 99% of the coronavirus transferred to the surface can be killed or inactivated by the bioactive material within 30 minutes after transfer to the surface. In other instances, at least 99% of the coronavirus transferred to the surface can be killed or inactivated by the bioactive material within 60 minutes after transfer to the surface. In some embodiments, at least 99% of the coronavirus transferred to the surface can be killed or inactivated by the bioactive material within 120 minutes after transfer to the surface.

In certain embodiments, the methods and handle covers or handles described herein can be used to prevent or reduce the spread of infections from one or more bacteria, including but not limited to, Bacillus, Pseudomonas, Bacteroides, Bordetella, Brucella, Campylobacter, Chlamydia, Clostridium, e.g., Clostridium difficile, Corynebacterium, Enterobacter, Enterococcus, Escherichia, Haemophilus, Klebsiella, Lactobacillus, Legionella, Listeria, Micrococcus, Mycobacterium, Mycoplasma, Neisseria, Rickettsia, Salmonella, Shigella, Staphylococcus, Streptococcus, Vibrio, Yersinia and other bacteria commonly encountered in a clinical setting. In certain examples, spores such as those from Bacillus species or Clostridium species, e.g., Clostridium difficile, can be inactivated. In some embodiments, the bioactive material can be effective to kill or inactivate one or more of Actinobacteria, Bacteriodetes, Firmicutes, Propionibacteriaceae, Lactobacillaceae and Proteobacteria as these bacteria are commonly encountered in public setting such as public restrooms and surfaces therein. If desired, the bioactive material can also be selected to kill or inactivate fungal organisms such as those commonly encountered in athletic facility showers, e.g., Tinea, Trichophyton, Candida and other fungal organisms.

In certain embodiments, any of the handle covers or handles described herein can be printed by applying suitable materials to a surface using a printer. The printer may be, for example, an inkjet printer, digital printer, laser printer, etc.

In certain instances, the handle covers or handles described herein can be used in combination with an antimicrobial agent or therapeutic. Illustrative antimicrobial agents include, but are not limited to, a sulfonamide, a trimethoprim-sulfamethoxazole, a quinolone, a fluoroquinolone, a quinone, a penicillin, a cephalosporin, a Beta-lactam antibiotic, a Beta-lactamase inhibitor, an aminoglycoside, a tetracycline, a chloramphenicol, an erythromycin, a macrolide, a clindamycin, isoniazid, rifampin, a pyrazinamide, an ethionamide, amphotericin B, imidazole, triazole, ketoconazole, miconazole, itraconazole, fluconazole, ciclopirox olamine, haloprogin, tolnaftate, naftifine, terbinafine, chloroquinone, and hydroxychloroquinone. Other antibacterial and antifungal agents could also be used. Combinations of two or more of any of these antimicrobial agents can also be used in combination with the handle covers or handles described herein.

In some embodiments, the handle covers or handles described herein can be used in combination with one or more antiviral agents or therapeutics including, but not limited to, Abacavir, Acyclovir, Adefovir, Amantadine, Ampligen, Amprenavir, Arbidol, Atazanavir, Atripla, Balavir, Baloxavir marboxil, Biktarvy, Boceprevir, Cidofovir, Cobicistat, Combivir, Daclatasvir, Darunavir, Delavirdine, Descovy, Didanosine, Docosanol, Dolutegravir, Doravirine, Ecoliever, Edoxudine, Efavirenz, Elvitegravir, Emtricitabine, Enfuvirtide, Entecavir, Etravirine, Famciclovir, Fomivirsen, Fosamprenavir, Foscarnet, Fosfonet, a fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Interferon, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, a protease inhibitor, Pyramidine, Raltegravir, Remdesivir, a reverse transcriptase inhibitor, Ribavirin, Rilpivirine (Edurant), Rimantadine, Ritonavir, Saquinavir, Simeprevir (Olysio), Sofosbuvir, Stavudine, Synergistic enhancer (antiretroviral), Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine and combinations thereof.

In some embodiments, an antimicrobial agent can be used in combination with an antiviral agent and one or more of the handle covers or handles described herein. For example, the antiviral can be used to treat a viral infection, an antimicrobial can be used to treat a secondary bacterial infection and the handle covers or handles described herein can be used to prevent or reduce spread of the virus and/or antimicrobials to third parties.

In some embodiments, the handle covers or handles described herein can be dispensed in a vending machine or other devices to permit addition of the handle covers or handles to another device by an end user. For example, the handle covers or handles can be placed in public places such as athletic fields, train stations, subway stations, airports, scooter or bike rental sites, etc. to permit a user to purchase and place the handle covers or handles on an article such as a handle or other devices. In some examples, the covers can be purchased and used by an end user when renting a personal transportation device such as, for example, an electric scooter, skateboard, bicycle, gasoline scooter, motorcycle, moped or other gasoline or electric powered vehicles.

Certain specific examples are described showing the articles and materials thereon can be used to kill or inactivate viruses and bioorganisms.

### EXAMPLE 1

A 8.5 inches by 11 inches sheet of material including a bioactive material comprising titanium dioxide doped with silver ions present in a surface coating was aseptically cut into 1″×1″ squares. Stainless steel control squares of the same size were ethanol sanitized and double rinsed in reverse osmosis prepared water and then autoclaved prior to use. Each of the test and control samples were placed into sterile Petri dishes using sterile forceps.

A stock vial of human coronavirus 229E (ATCC VR-740) was removed from cryo-storage and permitted to thaw. 0.010 mL aliquots were aseptically spread over the surface of each test and control square to ˜⅛ inch of the edge. Virus films were prepared in duplicate per test and control surface, per contact time (T=30 min, 1 hour, 2 hours and 4 hours). Control and test carrier were dried with Petri dish lids slightly ajar for 20 minutes at 24.7 degree Celsius, 36% relative humidity, Illuminance 1140 lux. Contact times were initiated when the control and test squares were visibly dry.

At the end of each contact time, the test and control carriers were aseptically transferred to tubes containing 2.0 mL of neutralizing solution (2% FBS EMEM). The carriers were vortexed for 30 seconds each to mechanically dislodged the microorganisms for enumeration. The inoculated sides of each carrier were further treater using a cell scraper to ensure adequate removal of the test viruses.

For cytotoxicity and neutralization effectiveness controls, one test and one control carrier each (with no virus film) were each aseptically transferred to neutralization tubes, and vortexed as described previously for the virus. The vortexed suspensions were serially diluted ten-fold in neutralizing solution, and selected dilutions were plated in quadruplicate onto the appropriate host cell monolayers (MRC-5, ATCC CCL-171) prepared to suitable confluency in multi-well trays. Virus control, cytotoxicity, neutralization validation, and sterility controls were performed concurrently. Virus reductions were calculated using the Spearman-Karber Method. Reference may be made to JIS Z 2801:2000. Antimicrobial Products—Test for Antimicrobial Activity and Efficacy. Japanese Standards Association. Tokyo, Japan.

No cytotoxicity was observed for the MRC-5 cells on the stainless steel control and tested squares.

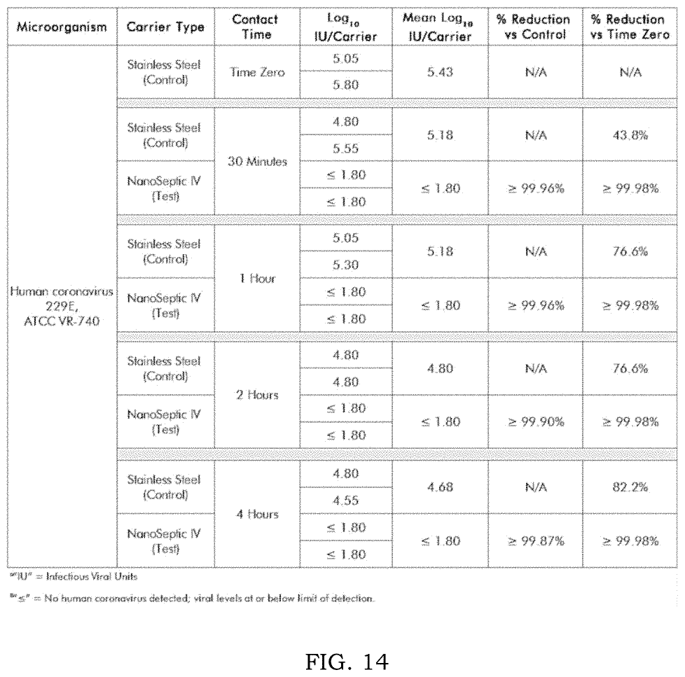

Referring to FIG. 14, as can be seen the test samples (labeled Nanoseptic IV), showed over a 99.96% reduction in viral for all measured times. In contrast, stainless steel control samples showed significantly less reduction at all measured times. These results are consistent with the tested samples being able to inactive the coronavirus and prevent infection of the MRC-5 human lung fibroblast cells.

### EXAMPLE 2

An article (2 inches by 2 inches) comprising a bioactive material comprising titanium dioxide doped with silver ions in a surface coating was tested for its ability to kill E. coli. using a modified ISO 22196 protocol. An overnight culture of E. coli cells (ATCC 8739) was diluted in sterile 1:500 Nutrient Broth. A sterile swab was dipped into the prepared test inoculum and used to inoculate each carrier via 13 passes (left to right=1 pass). Inoculated carriers were allowed to dry for 5 minutes followed by initiation of the contact time. Carriers were harvested after 5, 20, 60 and 120 minutes, vortexed to elute the viable bacteria and enumerated using standard dilution and pour plate techniques. Three replicates at each contact time were measured. Percent reduction was calculated as 100×(C−A)/C where A was the number of bacteria on the test carriers after the contact time and C is the number of bacteria on the control at time zero.

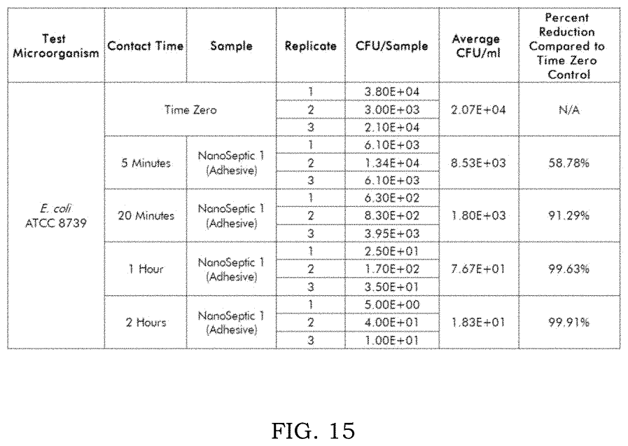

The results are shown in FIG. 15. At 20 minutes, a reduction in over 90% was observed. At 1 hour a reduction over 99% was observed. These results are consistent with the tested samples being able to kill the E. coli.

When introducing elements of the aspects, embodiments and examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations.

1. A handle cover comprising:
a release liner;
a first adhesive layer coupled to the release liner;
a substrate coupled to the first adhesive layer;
a second adhesive layer coupled to the substrate;
a carrier support material coupled to the second adhesive layer; and
a surface coating coupled to the carrier support material, the surface coating comprising a bioactive material to inactivate or kill bioorganisms that contact the surface coating.

2. The handle cover of claim 1, wherein the bioactive material comprises at least one of titanium, silver, copper and zinc.

3. The handle cover of claim 2, wherein the carrier support material comprises a polyurethane.

4. The handle cover of claim 1, wherein the bioactive material comprises a photocatalyst comprising titanium, and wherein the bioactive material comprises at least one additional transition metal.

5. The handle cover of claim 3, further comprising a second bioactive material embedded in the polyurethane of the carrier support material.

6. The handle cover of claim 5, wherein the bioactive material and the second bioactive material comprise different transition metals.

7. The handle cover of claim 3, wherein the first adhesive layer comprises a residue free adhesive.

8. The handle cover of claim 3, wherein the substrate material comprises a circular shape with memory.

9. The handle cover of claim 8, wherein the handle cover is optically transparent.

10. The handle cover of claim 9, wherein the substrate comprises a polyolefin.

11. A handle comprising:
an adhesive layer coupled to a handle substrate;
a carrier support material coupled to the adhesive layer; and
a surface coating coupled to the carrier support material, the surface coating comprising a bioactive material to inactivate or kill bioorganisms that contact the surface coating.

12. The handle of claim 11, wherein the bioactive material comprises at least one of titanium, silver, copper and zinc.

13. The handle of claim 12, wherein the carrier support material comprises a polyurethane.

14. The handle of claim 11, wherein the bioactive material comprises a photocatalyst comprising titanium, and wherein the bioactive material comprises at least one additional transition metal.

15. The handle of claim 13, further comprising a second bioactive material embedded in the polyurethane of the carrier support material.

16. The handle of claim 15, wherein the bioactive material and the second bioactive material comprise different transition metals.

17. The handle of claim 13, wherein the adhesive layer comprises a residue free adhesive.

18. The handle of claim 13, further comprising a tear off coupled to the surface coating, wherein the tear off comprises a second adhesive layer coupled to the surface coating, a second carrier support material coupled to the second adhesive layer, and a second surface coating coupled to the second carrier support material, the second surface coating comprising a bioactive material to inactivate or kill bioorganisms that contact the second surface coating.

19. The handle of claim 18, wherein the adhesive layer, the carrier support material and the surface coating are optically transparent.

20. The handle of claim 19, wherein the handle substrate comprises a polyolefin.

21-87. (canceled)
